(19) United States
(12) Patent Application Publication
(10) Pub. No.: US 2022/0241271 A1
(43) Pub. Date: Aug. 4, 2022

(54) INHALABLE DRY POWDERS

(71) Applicants: Joseph J. Guarneri, Stamford, CT (US); Chad C. Smutney, Watertown,, CT (US)

(72) Inventors: Joseph J. Guarneri, Stamford, CT (US); Chad C. Smutney, Watertown,, CT (US)

(21) Appl. No.: 17/593,503

(22) PCT Filed: Mar. 20, 2020
(86) PCT No.: PCT/US2020/023977

§ 371 Date: Sep. 20, 2021

Related U.S. Application Data
(60) Provisional application No. 62/822,303, filed on Mar. 22, 2019.

(51) Int. Cl. A61K 31/495; A61K 31/05; A61K 31/352; A61K 45/06; A61K 9/00; A61K 9/16

(52) U.S. Cl. CPC A61K 31/495; A61K 31/05; A61K 9/1617; A61K 45/06; A61K 9/0075; A61K 31/352

(57) ABSTRACT

The present disclosure relates to inhalable dry powder compositions comprising cannabinoids. In particular, the formulations are intended for use with dry powder inhalers for single use or multiple use comprising replaceable cartridges for delivery to the deep lung as medicinal agents. The inhalable dry powders are useful in the treatment of diseases and disorders, including pain.

### CROSS REFERENCE TO RELATED APPLICATIONS

This application is a 371 national stage application of PCT/US2020/023977, filed Mar. 20, 2020 claims the benefit of U.S. provisional patent application No. 62/822,303, filed on Mar. 22, 2019, and is also a continuation in part of U.S. patent application Ser. No. 16/601,440, filed Oct. 14, 2019 which is a continuation of U.S. patent application Ser. No. 15/418,388, filed on Jan. 27, 2017, which claims the benefit of U.S. Provisional Patent Application No. 62/289,095, filed Jan. 29, 2016, the entire disclosures of which are all incorporated herein by reference.

### TECHNICAL FIELD

The present disclosure relates to inhalable dry powder formulations comprising cannabinoids and methods of using them. In particular, the formulations are intended for use with dry powder inhalers for single use or multiple use with replaceable cartridges or capsules for delivery to the deep lung as medicinal agents.

### BACKGROUND

Drug delivery to lung tissue has been achieved using a variety of devices for inhalation, including nebulizers and inhalers, such as metered dose inhalers and dry powder inhalers to treat local disease or disorders. Dry powder inhalers used to deliver medicaments to the lungs contain a dose system of a powder formulation usually either in bulk supply or quantified into individual doses stored in unit dose compartments, like hard gelatin capsules/cartridges or blister packs. Bulk containers are equipped with a measuring system operated by the patient in order to isolate a single dose from the powder immediately before inhalation.

Dosing reproducibility with inhalers requires that the drug formulation is uniform and that the dose be delivered to a subject with consistency and reproducible results. Therefore, the dosing system ideally should operate to completely discharge all of the formulation effectively during an inspiratory maneuver when the patient is taking his/her dose. However, complete powder discharge from the inhaler is not required as long as reproducible dosing can be achieved. Flow properties of the powder formulation, and long term physical and mechanical stability in this respect, are more critical for bulk containers than they are for single unit dose compartments. Good moisture protection can be achieved more easily for unit dose compartments such as blisters. However, the materials used to manufacture the blisters allow air into the drug compartment and subsequently, the formulation loses viability with prolonged storage, particularly if the formulation to be delivered is hygroscopic. Ambient air permeating through the blisters carries in humidity that destabilizes the active ingredient. Additionally, dry powder inhalers which use blisters to deliver a medicament by inhalation can suffer with inconsistency of dose delivery to the lungs due to variations in geometry of the air conduit architecture resulting from puncturing films or peeling films of the blisters.

Dry powder inhalers can be breath activated or breath-powered and can deliver drugs by converting drug particles in a carrier into a fine dry powder which is entrained into an air flow and inhaled by the patient. Efficient drug delivery to the lungs and to the systemic circulation also depends, therefore and in-part, in the quality of the formulation to generate the aerosolized particles, the type of agents to be delivered and the delivery system used. Advantages of the lungs for delivery of systemic agents include the large surface area and the ease of uptake by the lung's mucosal surface.

Pulmonary drug delivery systems present many difficulties for use. For example, some devices use propellants, which can have deleterious effects to the user and compounds to be delivered; aerosolization of active agents including, but not limited to small molecules, biological agents such as proteins and peptides can lead to denaturation of their activities, and the device may have excessive loss of the agent/formulation to be delivered during aerozolization. One other problem associated with all of these forms of pulmonary drug delivery is that it is difficult to deliver drugs into the lungs due to problems in getting the drugs past all of the natural barriers, such as the cilia lining the trachea, and in trying to administer a uniform volume and weight of drug. Accordingly, there is room for improvement in pulmonary delivery of drugs in particular in the development of suitable inhalable formulations and effective delivery systems.

Cannabinoids have been discovered more recently to have numerous beneficial health effects and medicinal uses. For example, the Food and Drug Administration (FDA) recently approved an oral cannabinoid solution; Epidiolex® for treating seizures in Dravet syndrome (severe myoclonic epilepsy of infancy) and Lennox-Gastaut syndrome (LGS, severe childhood onset epilepsy) for patients who had previously failed multiple epilepsy medicinal treatments. Marinol (dronabinol) an oily resin in a capsule, is used as an antiemetic to control of nausea and vomiting caused by chemotherapeutic agents used in the treatment of cancer and to stimulate appetite in AIDS patients induced anorexia. Cesamet (nabilone) also is a synthetic cannabinoid provided in an oral capsule and used for controlling nausea and vomiting caused by chemotherapeutic agents used in the treatment of cancer. Controlling nausea and vomiting with orally-delivered capsules and solutions may not efficiently treat these symptoms.

Accordingly, new formulations or compositions and new routes of administration are needed to be developed in order to achieve consistent and reproducible delivery of the cannabinoids, for treating such conditions and many other conditions, and for facilitating administration of such drugs. Therefore, the inventors have identified the need to design and manufacture cannabinoid compositions for inhalation, which deliver consistent and effective doses of the cannabinoids with discrete and efficient dry powder inhalers.

### SUMMARY

The present disclosure is directed to dry powder compositions for pulmonary delivery comprising therapeutically effective amounts of a Cannabis derived molecule or cannabinoids for the treatment of diseases, disorders and conditions including cancer, epilepsy, eating disorders, and symptoms associated with treatment of disease, such as nausea and vomiting.

In particular embodiments, the compositions include, synthetic cannabinoids, naturally-occurring cannabinoids and/or extracted cannabinoids, and one or more pharmaceutically acceptable carriers suitable for pulmonary delivery, and/or pharmaceutically acceptable excipients. In some embodiments, the cannabinoid molecule can comprise an extracted naturally-occurring cannabinoid, or a synthetic cannabinoid, including, but not limited to tetrahydrocannabinol (THC) cannabidiol (CBD) and cannabinol and derivatives thereof, and combinations thereof.

In certain embodiments, the pharmaceutically acceptable carriers, include, diketopiperazines, including, fumaryl diketopiperazine; lipophilic compounds, including, phospholipids, including, 1,2-dipalmitoyl-sn-glycero-3-phosphocholine (DPPC) and 1,2-distearoyl-sn-glycero-3-phospho-choline (DSPC); sugars, including mannitol, lactose and xylitol; buffers, including phosphate, citrate and tartrate, and the like. In one embodiments, the content of phospholipid to be added to the composition depends on the type of phospholipid used. In one embodiment, the amount of phospholipid to be added can be up to 40% (w/w) of the composition.

In one embodiment, a dry powder pharmaceutical composition is provided comprising microcrystalline particles of a diketopiperazine, including, 3,6-bis(N-fumaryl-4-aminobutyl)diketopiperazine and an active agent, wherein the microcrystalline particles microcrystalline particles have an average pore size from about 23 nm to about 26.2 nm. In one embodiment, the dry powder pharmaceutical composition comprises microcrystalline particles having a surface area of ranges from about 59 m²/g to about 63 m²/g.

In some embodiments, the dry powder pharmaceutical composition comprises microcrystalline particles, wherein the porosity of the microcrystalline particles have average pore volumes of about 0.43 cm³/g and average pore size of 23 nm to about 28 nm.

In other embodiments herewith, a dry powder composition for delivering to the pulmonary tract and lungs includes, for example, a drug formulation for pulmonary delivery comprising a cannabinoid and a diketopiperazine in a crystalline form that self-assembles, an amorphous powder composition form, and/or a microcrystalline powder form comprising crystallites of the diketopiperazine that do not self-assemble in a suspension, or combinations thereof, and/or crystalline composite dry powders and the cannabinoid active agent. In alternate embodiments, the dry powder may be formulated in compositions further comprising other carriers and/or excipients other than diketopiperazines or in combination with the diketopiperazine, for example, a sugar, including, monito, xylitol, trehalose, and a cannabinoid active agent.

In certain embodiments, the dry powder compositions are provided in individual inhalers for single use. The cannabinoid compositions can also be provided in single use cartridges or capsules which are replaceable for use with multiple use inhalers. The cartridges and capsules comprising the dry powder for inhalation. The dry powders can be for local and/or systemic delivery into the pulmonary circulation.

In one embodiment, the dry powder inhaler is a breath-powered inhaler which is compact, reusable or disposable, has various shapes and sizes, and comprises a system of airflow conduit pathways for the effective and rapid delivery of powder medicament to the lungs and the systemic circulation. In some embodiments, the dry powder inhaler is provided with a container for holding the powder medicament for single use. In other embodiments, a multiple use inhaler can be provided with replaceable containers and/or cartridges. In these embodiments, the dry powder inhaler is configured to generate a resistance to flow value during an inhalation of about 0.05 to about 0.25 (√kPa)/liter per minute in use. In this and other embodiments, the dry powder inhaler can generate pressure differentials of at least 2 kPa and preferably from about 2 kPa to about 8 kPa for effective delivery of a dry powder dose.

In another embodiment, there is provided an inhalation system comprising a breath-powered dry powder inhaler, a cartridge or a capsule containing a dry powder for delivering to the pulmonary tract and lungs, including a dry powder cannabinoid medicament, wherein the medicament can comprise, for example, a drug formulation for pulmonary delivery such as a composition comprising a diketopiperazine in a crystalline form wherein the crystals self-assemble in suspension to make particles, an amorphous form, and/or a microcrystalline form comprising a crystalline composite powder wherein the crystals do not self-assemble in suspension, or combinations thereof, and an active agent. In alternate embodiments, the dry powder may, optionally, further comprise other carriers and/or excipients other than diketopiperazines, for example, a sugar, including, mannitol, lactose, trehalose, sorbitol, xylitol and an active agent. In some embodiments, the dry powder composition for use in the inhalation system comprises a diketopiperazine of the formula 3,6-bis(N-fumaryl-4-aminobutyl)-2,5-diketopiperazine.

In certain embodiments, the compositions can include a cannabinoid extracted from natural sources or synthetically made and can comprise from about 0.1% (w/w) to about 50% of the total weight of the composition or higher. In some embodiments the composition can include a cannabinoid from about 0.1% to about 10%, from about 0.5% to about 20%, from about 0.5% to about 50%, or from about 1% to about 75% of the total weight of the composition.

In one embodiment, a method of making a cannabinoid composition comprising precipitating diketopiperazine crystal particles in an aqueous suspension; washing the crystal particles in the precipitate by, for example, using tangential flow filtration; adding a cannabinoid to an ethanol solution; optionally filtering or winterizing the solution; removing a fatty acid layer; adding a phospholipid to the suspension comprising the diketopiperazing particles and spray drying the suspension to make bulk cannabinoid powder.

In particular embodiments, the inhalation system can be used, for example, in methods for treating conditions requiring localized or systemic delivery of a medicament, for example, in methods for treating disease and conditions, for example, including diseases and disorders including, cancer, glaucoma, HIV/AIDS, muscle spasms seizures, sever pain, severe nausea, cachexia or dramatic weight loss and muscle atrophy. In some embodiments, the method comprises, administering to a subject in need of treatment and inhaler comprising an effective amount of a composition comprising a diketopiperazine and a cannabinoid, and having the patient inhale at least once through said inhaler for at least 1-6 seconds, wherein the inhaler can be provided with a container, cartridge or a capsule in a dosing configuration for inhalation and for using the subject's own breath.

In one embodiment, a dose comprising a cannabinoid and a diketopiperazine can comprise from about 1 mg to about 20 mg of powder, or from about 1 mg to about 10 mg of powder in a high resistance inhaler. In one embodiment, the cannabinoid composition comprises at least 0.1 mg or at least 0.5 mg of the powder content for lung delivery.

In some embodiments, a dry powder drug delivery system is provided comprising a dry powder inhaler comprising a dry powder pharmaceutical composition comprising microcrystalline particles of 3,6-bis(N-fumaryl-4-aminobutyl)-2,5-diketopiperazine and a cannabinoid; wherein the cannabinoid is in an amount from 1% to 40% (w/w) of the total weight of the composition and the microcrystalline particles have average pore volumes of about 0.43 cm3/g and average pore size ranging from about 23.8 nm to 26.2 nm as determined by BJH adsorption.

In one embodiment, the dry powder inhalation system comprises a kit, including at least one of each of the components of the inhalation system, including an inhaler comprising the composition for treating the specific disease or disorder.

In certain embodiments, a method is provided for treating pain, including chronic pain in a patient, said method comprising: providing said patient an inhalation system comprising a dry powder inhaler and a pharmaceutical dry powder composition comprising a diketopiperazine and a cannabinoid and administering said dry powder composition to said patient by having the patient inhale deeply from said dry powder inhaler.

In alternate embodiments, the inhalation system comprises a drug delivery system for inhalation comprising a dry powder inhaler and a dry powder pharmaceutical composition; wherein the dry powder pharmaceutical composition comprises one of more of a cannabinoid, an antiviral agent, an anti-inflammatory, or an antibiotic compound, or combinations thereof. In one embodiment, the antiviral compound is for treating respiratory infections or disease caused by a virus. In certain embodiments, the antiviral compound is selected a salicylate such as acetylsalicylic acid, curcumin, and/or vitamin C. In one embodiment, the dry powder pharmaceutical composition can comprise an excipient acceptable for lung delivery, including, sugars such as lactose, mannitol, trehalose, xylitol; diketopiperazines and derivatives thereof, including, 3,6-bis(N-fumaryl-4-aminobutyl)-2,5-diketopiperazine; citrate; tartrate; or other pharmaceutical acceptable excipients and/or carriers or salts thereof. In one embodiment, the dry powder composition comprises 3,6-bis(N-fumaryl-4-aminobutyl)-2,5-diketopiperazine or a salt thereof, and curcumin in an amount up to 50 mg of powder by weight per dose.

In another embodiment, the dry powder composition comprises a diketopiperazine or a disodium salt thereof and curcumin alone and, optionally, acetylsalicylic acid of from 0.5 wt % to about 20 wt %, or from about 1 wt % to about 10 w % in the composition.

A method for treating viral disease, and in particular, respiratory track infections and lung infections comprising administering to a subject in need of treatment a therapeutic dose of a dry powder pharmaceutical composition comprising curcumin or derivative thereof, in an amount of 0.5 wt % to about 20 wt % in the dry powder pharmaceutical composition, and particles of 3,6-bis(N-fumaryl-4-aminobutyl)-2,5-diketopiperazine, and optionally, one or more pharmaceutical excipients or carriers. In another embodiment, the dry powder composition can further comprise a salicylate, including acetylsalicylic acid.

### DETAILED DESCRIPTION

In embodiments disclosed herein, dry powder compositions for use with dry powder inhalers for delivering dry powders including pharmaceutical medicaments to a subject by oral inhalation are described. In one embodiment, the compositions are for use with the dry powder inhaler which are breath-powered by an individual's inhalation effort, and are provided for single use as disposable inhalers or for multiple use with replaceable cartridges. In an exemplary embodiment, the inhaler can be designed for single use wherein the cannabinoid formulation is provided contained within the inhaler and can be accessed for inhalation by activating the inhaler into a dosing configuration manually. In an exemplary embodiment of a multiple use inhaler, the inhaler is provided empty and a capsule or a cartridge containing the cannabinoid composition is placed/mounted into the inhaler and the container is configured for inhalation automatically upon insertion or thereafter by a mechanism provided within the inhaler. For example, a carrier mechanism which upon closing the inhaler creates an air pathway within cartridge and accessible by the inhaler air conduits. In one embodiment, the capsule or cartridge contains an inhalable cannabinoid dry powder, including but not limited to pharmaceutical formulations.

The dry powder inhalers are provided in various embodiments of shapes and sizes, and can be reusable, easy to use, inexpensive to manufacture and/or produced in high volumes in simple steps using plastics or other acceptable materials. Various embodiments of the dry powder inhalers are provided herein and in general, the inhalation systems comprise inhalers, powder-filled cartridges.

As used herein, the term “disposable inhaler” is an inhaler provided with a dose of powder for a one-time use. The inhaler is discarded after a single use or after inhalation of its powder content.

As used herein, the term “a unit dose inhaler” refers to an inhaler that is adapted to receive a single cartridge or container comprising a dry powder formulation and delivers a single dose of a dry powder formulation by inhalation from a single container to a user. It should be understood that in some instances multiple unit doses will be required to provide a user with a specified dosage. In one embodiment, the unit dose inhaler can be used multiple times.

As used herein a “cartridge” is an enclosure configured to hold or contain a dry powder formulation, a powder containing enclosure, which has a cup or container and a lid. The cartridge is made of rigid materials, and the cup or container is moveable relative to the lid in a translational motion or vice versa.

As used herein a “powder mass” is referred to an agglomeration of powder particles or agglomerate having irregular geometries such as width, diameter, and length.

As used herein a “unit dose” refers to a pre-metered dry powder formulation for inhalation. Alternatively, a unit dose can be a single container having multiple doses of formulation that can be delivered by inhalation as metered single amounts. A unit dose cartridge/container contains a single dose. Alternatively it can comprise multiple individually accessible compartments, each containing a unit dose.

As used herein, the term “about” is used to indicate that a value includes the standard deviation of error for the device or method being employed to determine the value.

As used herein, the term “microparticle” refers to a particle with a diameter of about 0.5 to about 1000 μm, irrespective of the precise exterior or interior structure. Microparticles having a diameter of between about 0.5 and about 10 microns can reach the lungs, successfully passing most of the natural barriers. A diameter of less than about 10 microns is required to navigate the turn of the throat and a diameter of about 0.5 μm or greater is required to avoid being exhaled. To reach the deep lung (or alveolar region) where most efficient absorption is believed to occur, it is preferred to maximize the proportion of particles contained in the “respirable fraction” (RF), generally accepted to be those particles with an aerodynamic diameter of about 0.5 to about 6 μm, though some references use somewhat different ranges, as measured using standard techniques, for example, with an Anderson Cascade Impactor. Other impactors can be used to measure aerodynamic particle size such as the NEXT GENERATION IMPACTOR™ (NGI™, MSP Corporation), for which the respirable fraction is defined by similar aerodynamic size, for example <6.4 μm. In some embodiments, a laser diffraction apparatus is used to determine particle size, for example, the laser diffraction apparatus disclosed in U.S. Pat. No. 8,508,732, which disclosure is incorporated herein in its entirety for its relevant teachings related to laser diffraction, wherein the volumetric median geometric diameter (VMGD) of the particles is measured to assess performance of the inhalation system. For example, in various embodiments cartridge emptying of ≥80%, 85%, or 90% and a VMGD of the emitted particles of <12.5 μm, <7.0 μm, or <4.8 μm can indicate progressively better aerodynamic performance.

Respirable fraction on fill (RF/fill) represents the percentage (%) of powder in a dose that is emitted from an inhaler upon discharge of the powder content filled for use as the dose, and that is suitable for respiration, i.e., the percent of particles from the filled dose that are emitted with sizes suitable for pulmonary delivery, which is a measure of microparticle aerodynamic performance. As described herein, a RF/fill value of 40% or greater than 40% reflects acceptable aerodynamic performance characteristics. In certain embodiments disclosed herein, the respirable fraction on fill can be greater than 50%. In an exemplary embodiment, a respirable fraction on fill can be up to about 80%, wherein about 80% of the fill is emitted with particle sizes <5.8 μm as measured using standard techniques.

As used herein, the term “dry powder” refers to a fine particulate composition that is not suspended or dissolved in a propellant, or other liquid. It is not meant to necessarily imply a complete absence of all water molecules.

As used herein, “amorphous powder” refers to dry powders lacking a definite repeating form, shape, or structure, including all non-crystalline powders.

In exemplary embodiments herewith, the present devices can be manufactured by several methods and from various materials. In one embodiment, the inhalers and cartridges are made, for example, by injection molding techniques, thermoforming, blow molding, pressing, 3D printing, and the like using various types of plastic materials, including, polypropylene, cyclicolephin co-polymer, nylon, and other compatible polymers and the like. In certain embodiments, the dry powder inhaler can be assembled using top-down assembly of individual component parts. In some embodiments, the inhalers are generally provided in compact sizes, for example, from about 1 inch to about 5 inches in dimension, and generally, the width and height are less than the length of the device. In certain embodiments the inhaler is provided in various shapes including, relatively rectangular bodies, although other shapes can be used such as cylindrical, oval, tubular, squares, oblongs, and circular forms.

In embodiments described and exemplified herewith, the inhalers effectively fluidize, deagglomerate or aerosolize a dry powder formulation by using at least one relatively rigid flow conduit pathway for allowing an airflow to enter the inhaler. For example, the inhaler is provided with a first air flow pathway for entering and exiting a cartridge containing the dry powder, and a second air pathway which can merge with the first air flow pathway exiting the cartridge. The flow conduits, for example, can have various shapes and sizes depending on the inhaler configuration. In one embodiment, the inhaler are high resistance inhalers resistance value of, for example, approximately 0.055 to about 0.250 (√kPa)/liter per minute. Therefore, in the system, peak inhalation pressure drops of between 2 and 20 kPa produce resultant peak flow rates of about between 7 and 70 liters per minute. These flow rates result in greater than 75% of the cartridge contents dispensed in fill masses between 1 and 50 mg. In some embodiments, these performance characteristics are achieved by end users within a single inhalation maneuver to produce cartridge dispense percentage of greater than 90% of the powder contained in a cartridge.

In embodiments disclosed herein, a dry powder formulation can consist of a crystalline powder, an amorphous powder, or combinations thereof, wherein the powder is dispensed with consistency from the inhaler in less than about 2 seconds. The present inhaler system has a high resistance value of approximately 0.065 to about 0.200 (√kPa)/liter per minute. Therefore, in the system comprising a cartridge, peak inhalation pressure drops applied of between 2 and 20 kPa produce resultant peak flow rates of about through the system of between 7 and 70 liters per minute. These flow rates result in greater than 75% of the cartridge contents dispensed in fill masses between 1 and 30 mg, or up to 50 mg of powder. In some embodiments, these performance characteristics are achieved by end users within a single inhalation maneuver to produce cartridge dispense percentage of greater than 90%. In certain embodiments, the inhaler and cartridge system are configured to provide a single dose by discharging powder from the inhaler as a continuous flow, or as one or more pulses of powder delivered to a patient. In an embodiment, an inhalation system for delivering a dry powder formulation to a patient's lung(s) is provided, comprising a dry powder inhaler configured to have flow conduits with a total resistance to flow in a dosing configuration ranging in value from 0.065 to about 0.200 (√kPa)/liter per minute. In this and other embodiments, the total resistance to flow of the inhalation system is relatively constant across a pressure differential range of between 0.5 kPa and 7 kPa.

The structural configuration of the inhaler allows the deagglomeration mechanism to produce respirable fractions greater than 50% and particles of less than 5.8 μm. The inhalers can discharge greater than 85% of a powder medicament contained within a container during an inhalation maneuver. Generally, the inhalers herein depicted herewith can discharge greater that 90% of the cartridge contents or container contents in less than 3 seconds at pressure differentials between 2 and 5 kPa with fill masses ranging up to 30 mg or 50 mg.

While the present inhalers are primarily described as breath-powered, in some embodiments, the inhaler can be provided with a source for generating the pressure differential required to deagglomerate and deliver a dry powder formulation. For example, an inhaler can be adapted to a gas powered source, such as compressed gas stored energy source, such as from a nitrogen can, which can be provided at the air inlet ports. A spacer can be provided to capture the plume so that the patient can inhale at a comfortable pace.

In embodiments described herewith, the inhaler can be provided as a reusable inhalers for delivering a single unit dose. A reusable inhaler means that it can be used multiple times which can be predetermined depending on the formulation to be delivered and discarded once it has reached its maximal usage.

Devices and systems useful in pulmonary delivery of present powders exhibit a wide range of characteristics. Embodiments include systems comprising an inhaler, an integral or installable unit dose cartridge comprising the desirable powder doses. Pulmonary delivery of powders include carriers and excipients which facilitate effective delivery of the cannabinoids as active agents to the lungs. An exemplary embodiment is fumaryl diketopiperazine, also known as 3,6-bis(N-fumaryl-4-aminobutyl)-2,5-diketopiperazine; FDKP.

Dry powders manufactured using diketopiperazines can be made by lyophilizing, or spray-drying solution, or suspensions of the various desired cannabinoid formulations made by various techniques. In one embodiment, diketopiperazine-based crystalline microparticles with a specific surface area (SSA) of between about 15 m²/g and about 67 m²/g exhibit characteristics beneficial to delivery of drugs to the lungs such as improved aerodynamic performance and improved drug adsorption. In some embodiments, high capacity crystalline FDKP microparticles for use in formulations containing certain molecules, for example, have a specific surface area which is less than 35 m²/g and specific surface area of these particles can range from about 19 m²/g to about 30 m²/g or from about 28 m²/g to about 71 m²/g, or from about 19 m²/g to about 57 m²/g depending on the amount of active agent.

In one embodiment, the dry powder medicament may comprise, for example, a diketopiperazine and a pharmaceutically active ingredient. In this embodiment, the pharmaceutically active ingredient or active agent can be any type depending on the disease or condition to be treated. In another embodiment, the diketopiperazine can include, for example, symmetrical molecules and asymmetrical diketopiperazines having utility to form particles, microparticles and the like, which can be used as carrier systems for the delivery of active agents to a target site in the body. The term “active agent” is referred to herein as the therapeutic agent, or molecule, including, small molecules, including neurotransmitters including cannabinoids can be encapsulated, associated, joined, complexed or entrapped within or adsorbed onto the diketopiperazine particles in forming the formulation. Any cannabinoid form can be combined with a diketopiperazine. The drug delivery system can be used to deliver the active agents for therapeutic, prophylactic, or diagnostic use.

The fumaryl diketopiperazine 3,6-bis(N-fumaryl-4-aminobutyl)-2,5-diketopiperazine; FDKP is one preferred diketopiperazine for pulmonary applications:

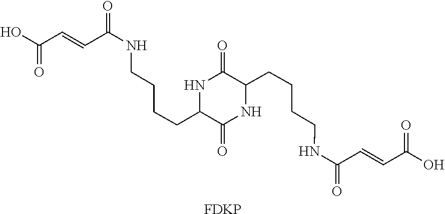 [SMILES: O=C(O)/C=C/C(=O)NCCCCC1NC(=O)C(CCCCNC(=O)/C=C/C(=O)O)NC1=O ; "FDKP"]

Microparticles for pulmonary delivery having a diameter of between about 0.5 and about 10 μm can reach the lungs and can reach the systemic circulation and deliver an active agent. A diameter of less than about 10 μm is required to navigate the turn of the throat and a diameter of about 0.5 μm or greater is required to avoid being exhaled. Generally, microparticles having diameters greater than 10 μm or greater than 20 μm are useful for local delivery to the respiratory tract and lungs.

Microparticles having a diameter of less than 10 microns can reach the lungs successfully passing most of the natural barriers within the respiratory tract. Embodiments disclosed herein show that microparticles with a specific surface area (SSA) of between about 15 m²/g and about 75 m²/g or from about 30 m²/g to about 71 m²/g exhibit characteristics beneficial to delivery of drugs to the lungs such as improved aerodynamic performance and improved drug adsorption. In some embodiments herewith, there is provided a composition comprising crystalline fumaryl diketopiperazine (FDKP) microparticles having a specific trans isomer content of about 35 to about 65%, or 45 to about 63%, or 45 to about 60%.

In exemplary embodiments, a diketopiperazine based composition for pulmonary delivery is provided with a cannabinoid active agent, wherein the diketopiperazine is fumaryl diketopiperazine and comprises a plurality of substantially uniformly formed, microcrystalline particles, wherein the particles have a substantially hollow spherical structure and comprise a shell comprising crystallites of a diketopiperazine, that do not self-assemble in suspension when formed, and the particles have a volumetric mean geometric diameter less than equal to 5 μm; wherein the particles are formed by a method comprising the step of combining diketopiperazine in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus. In some embodiments, the composition comprising fumaryl diketopiperazine and a cannabinoid comprises particles further comprising a phospholipid prior to spray drying the suspension.

In some embodiments, a diketopiperazine-based composition for pulmonary delivery is provided with an active agent, wherein the diketopiperazine is a salt of fumaryl diketopiperazine, including sodium, magnesium, and the composition comprises the amorphous powder.

A system for the delivery of an inhalable dry powder is also provided, comprising: a) a dry powder comprising a cannabinoid medicament, and b) an inhaler comprising a powder containing cartridge, the cartridge comprising a gas inlet and a gas outlet, and a housing in which to mount the cartridge and defining two flow pathways, a first flow pathway allowing gas to enter the gas inlet of the cartridge, a second flow pathway allowing gas to bypass the enclosure gas inlet, and a mouthpiece and upon applying a pressure drop of ≥2 kPa across the inhaler plume of particles is emitted from the mouthpiece wherein 50% of said emitted particles have a VMAD of ≤10 μm, wherein flow bypassing the cartridge gas inlet is directed to impinge upon the flow exiting the enclosure substantially perpendicular to the gas outlet flow direction.

The present disclosure also provides dry powder compositions comprising crystalline particles compositions, improved microcrystalline particles, or compositions comprising amorphous powders, methods of making the particles, and methods that allow for improved delivery of drugs to the lungs for treating diseases and disorders in a subject. Embodiments disclosed herein achieve improved delivery by providing crystalline diketopiperazine compositions comprising microcrystalline diketopiperazine particles having high capacity for drug adsorption yielding powders having high drug content of one or more active agents. Powders made with the present microcrystalline particles can deliver increased drug content in lesser amounts of powder dose, which can facilitate drug delivery to a patient. The powders can be made by various methods including, methods utilizing surfactant-free solutions or solutions comprising surfactants depending on the starting materials.

In alternate embodiments disclosed herein can comprise a dry powder for inhalation comprising a plurality of substantially uniform, microcrystalline particles, wherein the microcrystalline particles can have a substantially hollow spherical structure and comprise a shell which can be porous, comprising crystallites of a diketopiperazine that do not self-assemble in a suspension or in solution. In certain embodiments, the microcrystalline particles can be substantially hollow spherical and substantially solid particles comprising crystallites of the diketopiperazine depending on the drug and/or drug content provided and other factors in the process of making the powders. In one embodiment, the microcrystalline particles comprise particles that are relatively porous, having average pore volumes of about 0.43 cm³/g, ranging from about 0.4 cm³/g to about 0.45 cm³/g, and average pore size ranging from about 23 nm to about 30 nm, or from about 23.8 nm to 26.2 nm as determined by BJH adsorption.

Certain embodiments disclosed herein comprises powders comprising a plurality of substantially uniform, microcrystalline particles, wherein the particles have a substantially spherical structure comprising a shell which can be porous, and the particles comprise crystallites of a diketopiperazine that do not self-assemble in suspension or solution, and have a volumetric median geometric diameter less than 5 μm; or less than 2.5 μm.

In a particular embodiment herein, up to about 92% of the microcrystalline particles have a volumetric median geometric diameter of 5.8 μm. In one embodiment, the particle's shell is constructed from interlocking diketopiperazine microcrystals having one or more drugs adsorbed on their surfaces. In some embodiments, the particles can entrap the drug in their interior void volume and/or combinations of the drug adsorbed to the crystallites' surface and drug entrapped in the interior void volume of the spheres.

In certain embodiments, a diketopiperazine composition comprising a plurality of substantially uniformly formed, microcrystalline particles is provided, wherein the particles have a substantially hollow spherical structure and comprise a shell comprising crystallites of a diketopiperazine that do not self-assemble; wherein the particles are formed by a method comprising the step of combining diketopiperazine having a trans isomer content ranging from about 45% to 65% in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus.

The method can further comprise the steps of adding with mixing a solution comprising an active agent or an active ingredient such as a drug or bioactive agent prior to the spray drying step so that the active agent or active ingredient is adsorbed and/or entrapped on or within the particles. Particles made by this process can be in the submicron size range prior to spray-drying.

In certain embodiments, a diketopiperazine composition comprising a plurality of substantially uniformly formed, microcrystalline particles is provided, wherein the particles have a substantially hollow spherical structure and comprise a shell comprising crystallites of a diketopiperazine that do not self-assemble, and the particles have a volumetric mean geometric diameter less than equal to 5 μm; wherein the particles are formed by a method comprising the step of combining diketopiperazine in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus.

The method can further comprise the steps of adding with mixing a solution comprising a cannabinoid active agent dissolved in an alcohol solution and the diketopiperazine prior to the spray drying step so that the active agent or active ingredient is adsorbed and/or entrapped on or within the particles. Particles made by this process can be in the submicron size range prior to spray-drying.

In certain embodiments, a diketopiperazine composition comprising a plurality of substantially uniformly formed, microcrystalline particles is provided, wherein the microcrystalline particles have a substantially hollow spherical structure and comprise a shell comprising crystallites of a diketopiperazine that do not self-assemble, and the particles have a volumetric mean geometric diameter less than equal to 5 μm; wherein the particles are formed by a method comprising the step of combining diketopiperazine in a solution and a solution of acetic acid without the presence of a surfactant and without the presence of an active agent, and concurrently homogenizing in a high shear mixer at high pressures of up to 2,000 psi to form a precipitate; washing the precipitate in suspension with deionized water; concentrating the suspension and drying the suspension in a spray drying apparatus.

In certain embodiments wherein the starting material comprising the active ingredient is an extract exhibiting a high degree of viscosity, or a substance having a honey like viscous appearance, the microcrystalline particles are formed as above and by washing them in water using tangential flow filtration prior to combining with the extract or viscous material. After washing in water, the resultant particle suspension is lyophilized to remove the water and re-suspended in an alcohol solution, including ethanol or methanol prior to adding the active ingredient as a solid, or in a suspension, or in solution. In one embodiment, optionally, the method of making the composition comprises the step of adding any additional excipient, including one or more, amino acid, such as leucine, isoleucine, norleucine, methionine or one or more phospholipids, for example, 1,2-dipalmitoyl-sn-glycero-3-phosphocholine (DPPC) or 1,2-distearoyl-sn-glycero-3-phosphocholine (DSPC), concurrently with the active ingredient or subsequent to adding the active ingredient, and prior to spray drying. In certain embodiments, formation of the composition comprises the step wherein the extract comprising desired cannabinoid active agent is optionally filtered, or winterized to separate and remove layers of unwanted materials such as lipids to increase its solubility.

The method can further comprise the steps of adding while mixing a first solution comprising a diketopiperazine; adding a second solution comprising DPPC or DSPC, mixing the solutions, which solution can optionally be performed with or without homogenization in a high shear mixer, adding and mixing a third solution comprising a cannabinoid active agent, including a cannabidiol, or THC prior to the spray drying step so that the active agent is adsorbed and/or entrapped on or within the particles. Particles made by this process can be in the submicron, crystals size range prior to spray-drying, or the particles can be formed from the solution during spray-drying. The spray-dried powder comprises substantially homogenous particles which are low in density and require very little energy to deagglomerate.

In some embodiments herewith, the drug content can be delivered on crystalline powders using FDKP and which are lyophilized or sprayed dried at contents up to about 10%, or up to about 20%, or up to about 30% or higher. In embodiments using microcrystalline particles formed from FDKP, or FDKP disodium salt, and wherein the particles do not self-assemble and comprise submicron size particles, drug content can typically be greater than 0.01% (w/w). In one embodiment, the drug content to be delivered with the microcrystalline particles of from about 0.01% (w/w) to about 75% (w/w); from about 1% to about 50% (w/w), from about 10% (w/w) to about 25% (w/w), or from about 10% to about 20% (w/w), or from 5% to about 30%, or greater than 25% depending on the drug to be delivered. An example embodiment wherein the drug is cannabidiol or THC, the present microparticles typically comprise approximately 5% to 45% (w/w), or from about 10% to about 20% (w/w) or higher of the total content of the composition. In certain embodiments, the drug content of the particles can vary depending on the form and size of the drug to be delivered. In some embodiments, the density of bulk powder, or bulk density comprising FDKP in microcrystalline or crystalline composite form can be less than about 0.2 g/L. In certain embodiments, the bulk density can range from about 0.05 g/L to about 0.15 g/mL, or from 0.10 g/L to about 0.15 g/L. In one embodiment, the microcrystalline composite particles of spray-dried powders have a specific surface area of from about 30 m²/g to about 60 m²/g.

In an embodiment, the compositions for delivering with the inhalers herein can comprise fumaryl diketopiperazine crystalline particles or crystalline composite particles, and an active agent such as cannabinoids, including, tetrahydrocannabinol (THC) and/or cannabidiol. In composition wherein a cannabinoid is used as an active agent, the cannabinoid, including, derivatives and/or analog thereof content can be up to 40% (w/w) or higher with powder delivery greater than 40% and up to 99% of the inhaler content. In some embodiments, the cannabinoid and/or other active agent content in the composition can range from about 0.1% to about 40 wt %, from about 1% to about 30%, from about 5% to about 25% (w/w) of the powder content or higher. The compositions herein can also comprise one or more excipients including amino acids such as leucine, isoleucine, methionine and the like and one or more phospholipids, for example, 1,2-dipalmitoyl-sn-glycero-3-phosphocholine (DPPC) or 1,2-distearoyl-sn-glycero-3-phosphocholine (DSPC) prior to spray drying in amounts up to about 25% (w/w), ranging from about 1% (w/w) to about 25%, or 2.5% to 20% (w/w), or 5% to 15% (w/w). In this embodiment, the inhalers can discharge from about 50% to 100% of the composition of up to 50 mg of powder in a single inhalation. In this embodiment, the compositions can be administered to a subject in need of treatment as needed. A dose of powder containing active ingredients can be provided in one or more inhalations through the dry powder inhaler.

In one embodiment, cannabinoid compositions can be made comprising fumaryl diketopiperazine disodium salt, or crystalline composite particles of fumaryl diketopiperazine and an excipient, including, an amino acid such as leucine, isoleucine or methionine to improve storage stability to the composition. In this embodiment, the cannabinoid inhalable composition can be used in the prevention and treatment of chemotherapy-induced nausea and vomiting by self-administering the powder in a single inhalation using an inhaler comprising a dose of the composition from about 5 to 30 minutes and preferably from about 5 to 15 minutes prior to or concurrently with the patient receiving the dose of the chemotherapy.

In alternate embodiments, the pharmaceutically acceptable carrier for making dry powders can comprise any carriers or excipients useful for making dry powders and which are suitable for pulmonary delivery. Example of suitable carriers and excipients include, sugars, including saccharides and polysaccharides, such as lactose, mannose, sucrose, mannitol, trehalose; citrates, amino acids such as glycine, L-leucine, isoleucine, trileucine, tartrates, methionine, vitamin A, vitamin E, zinc citrate, trisodium citrate, zinc chloride, polyvinylpyrrolidone, polysorbate 80, phospholipids including diphosphotidylcholine and the like. In a particular embodiment, the pharmaceutical composition comprises a diketopiperazine

In still yet a further embodiment, a method of treating nausea, vomiting, and pain that can be associated with a disease is disclosed. The method comprises administering to a patient in need of therapy an inhalable dry powder composition or formulation comprising, for example, a diketopiperazine having the formula 2,5-diketo-3,6-di(4-X-aminobutyl)piperazine, wherein X is selected from the group consisting of succinyl, glutaryl, maleyl, and fumaryl and a cannabinoid. In this embodiment, the dry powder composition is a pharmaceutical composition which can comprise a diketopiperazine salt. In one embodiment, there is provided a dry powder pharmaceutical composition or formulation, wherein the diketopiperazine is 2,5-diketo-3,6-di-(4-fumaryl-aminobutyl)piperazine, with or without a pharmaceutically acceptable carrier, or excipient and a cannabinoid.

Exemplary cannabinoids, include, tetrahydrocannabinol (THC) cannabidiol (CBD) and cannabinol. In a particular embodiment, the pharmaceutical composition comprises 3-6-bis(4-fumaryl-4-aminobutyl)-2,5-diketopiperazine; tetrahydrocannabinol, cannabidiol, or cannabinol, or a combination thereof; and 1,2-dipalmitoyl-sn-glycero-3-phosphocholine (DPPC) or 1,2-distearoyl-sn-glycero-3-phosphocholine in an amount up to about 105 or up to about 15% (w/w), and optionally, an amino acid, including leucine, isoleucine, or methionine. In certain embodiments, the pharmaceutical composition can comprise a sugar, including, mannitol and lactose. The pharmaceutical composition can further comprise a surfactant, including, polysorbate 80 in an amount ranging from 0.05% to about 3% (w/w), or from about 1% to about 2% (w/w) of the total content of the composition. In some embodiments, the compositions can also comprise other pharmaceutically acceptable carriers and/or excipients, including, polyvinyl pyrrolidone, and polyethylene glycol from about 0.5% to about 6%.

An inhalation system for delivering a dry powder formulation to a patient's lung(s) is provided, wherein the system comprises a dry powder inhaler configured to have flow conduits with a total resistance to flow in a dosing configuration ranging in value from 0.055 to about 0.200 (√kPa)/liter per minute.

In one embodiment, a dry powder inhalation kit is provided comprising a dry powder inhaler as described above, one or more medicament cartridges comprising a dry powder formulation for treating a chronic pain as a result of a disorder or disease, including, cancer and epilepsy.

In one embodiment, a method of self-administering a dry powder formulation to one's lung(s) with a dry powder inhalation system is also provided. The method comprises: obtaining a dry powder inhaler in a closed position and having a mouthpiece; obtaining a cartridge comprising a pre-metered dose of a dry powder formulation in a containment configuration; opening the dry powder inhaler to install the cartridge; closing the inhaler to effectuate movement of the cartridge to a dose position; placing the mouthpiece in one's mouth, and inhaling once deeply to deliver the dry powder formulation.

Methods of treating a disease or disorder in a patient with the dry powder inhaler embodiments disclosed herewith is also provided. The method of treatment comprises providing to a patient in need of treatment a dry powder inhaler comprising a cartridge containing a dose of an inhalable formulation comprising an active ingredient selected from the group as described above and a pharmaceutical acceptable carrier and/or excipient; and administering the inhalable formulation by having the patient inhale through the dry powder inhaler deeply for about 3 to 4 seconds to deliver the dose. In the method, the patient can resume normal breathing pattern thereafter.

In one embodiment, the method comprises administering to a patient in need of treatment for relief of chronic pain, improve sleep, alleviate neuropathy, and/or reduce anxiety an inhalable dry powder pharmaceutical composition comprising particles of a diketopiperazine and a cannabinoid selected from tetrahydrocannabinol, cannabidiol and cannabinol. In one embodiment the diketopiperazine is 3,6-bis(4-fumaryl-4-aminobutyl)-2,5-diketopiperazine.

In certain embodiments, a method is provided for treating pain in a patient, said method comprising: providing said patient an inhalation system comprising a dry powder inhaler and a pharmaceutical dry powder composition comprising a diketopiperazine and a cannabinoid and administering said dry powder composition to said patient by having the patient inhale deeply from said dry powder inhaler. The dry powder pharmaceutical compositions may further comprise one or more anti-inflammatory agents, and other active agents, for example, acetylsalicylic acid and derivatives thereof, acetaminophen.

In alternate embodiments, the inhalation system comprises a drug delivery system for inhalation comprising a dry powder inhaler and a dry powder composition; wherein the dry powder composition comprises an antiviral or an antibiotic compound. In one embodiment, the antiviral compound is for treating respiratory infections or disease caused by a virus. In certain embodiments, the antiviral compound is selected a salicylate such as acetylsalicylic acid, curcumin, and/or vitamin C. In one embodiment, the dry powder pharmaceutical composition can comprise an excipient acceptable for lung delivery, including, sugars such as lactose, mannitol, trehalose, xylitol; diketopiperazines and derivatives thereof, including, 3,6-bis(N-fumaryl-4-aminobutyl)-2,5-diketopiperazine; citrate; tartrate; or other pharmaceutical acceptable excipients and/or carriers or salts thereof. In one embodiment, the dry powder composition comprises 3,6-bis(N-fumaryl-4-aminobutyl)-2,5-diketopiperazine or a salt thereof, and curcumin in an amount up to 50 mg of powder by weight per dose.

In an exemplary embodiment, the dry powder composition comprises a diketopiperazine, for example, 3,6-(N-fumaryl-4-aminobutyl)-2,5-diketopiperazine, or a disodium salt thereof and curcumin, analog or derivative thereof or combinations thereof and, optionally, acetylsalicylic acid of from 0.1 wt % to about 80 wt %, or from about 1 wt % to about 20 w % in the composition. In this embodiment, the dry powder composition can comprise other pharmaceutically acceptable excipients, for example, a phospholipid, including, DSPC, DPPC and the like; a sugar, including mannitol, trehalose, lactose, xylitol; amino acid, including, leucine, isoleucine, methionine, glycine, and the like.

A method for treating viral disease, and in particular, respiratory tract infections and lung infections comprising administering to a subject in need of treatment a therapeutic dose of a dry powder pharmaceutical composition comprising curcumin or derivative thereof, in an amount of 0.5 wt % to about 20 wt % in the dry powder pharmaceutical composition, and particles of 3,6-bis(N-fumaryl-4-aminobutyl)-2,5-diketopiperazine, and optionally, one or more pharmaceutical excipients or carriers. In another embodiment, the dry powder composition can further comprise a salicylate.

In one embodiment, a method of treating a viral infection, including, a retroviral infection is provided, the method comprising: providing a subject a dry powder inhaler comprising a pharmaceutical composition comprising a diketopiperazine including, 3,6-bis(N-fumaryl-4-aminobutyl)-2,5-diketopiperazine, and one or more antiviral agents, including, ribavirin, siRNA, acyclovir, amantidine, forscarnet, glancyclovir, oseltamivir, valacyclovir, zidarabine, zanamivir, abacavir, amprenavir, didanosine, indinavir, efavirenz, lamivudine, lopinavir, stabudine, nelfinavir, saquinovir, zalcitabine, zudivudine, and the like. The dry powder pharmaceutical composition may further comprise one or more pharmaceutically acceptable excipients and/or carriers, including, phospholipids, sugars, as defined above.

In another embodiments, the method for treatment of a subject as a prophylaxis or for treating respiratory disease comprises administering to a subject in need a therapeutic amount of a dry powder pharmaceutical composition comprises 3,6-bis(N-fumaryl-4-aminobutyl)-2,5-dikepiperazine particles and one or more active agents, including quinine derivates and salts thereof, including, chloroquine, mefloquine, primaquine, tafenoquine, hydroxychloroquine or salt thereof, including, hydroxychloroquine sulfate; acetyl salicylic acid, analogs or derivatives therefor, and one or more pharmaceutically acceptable excipients. The active ingredient can be provided by oral inhalation using a breath powered dry powder inhaler for single use or multiple use; wherein the composition is provided in a capsule or cartridge.

In alternate embodiments, a method of treating a respiratory tract infection and infections of the lungs is provided, which method comprises providing a subject in need of treatment an inhalation system comprising a dry powder inhaler and a dry powder pharmaceutical composition comprising a diketopiperazine, one or more active agents and one or more pharmaceutically acceptable excipients; wherein the active agents or combinations thereof, include, antibiotics, antivirals, anti-inflammatories, anti-asthmatics. In one embodiment, the antibiotic can be selected from azithromycin, tobramycin, doxycycline, minocycline, tetracyclin, ciproflaxin, amoxicillin, penicillin, ceftin, ceftriaxone, cephalexin, fosfomycin, clindamycin, levofloxacin, nalidixic acid, nitrofurantoin, tgrimethoprim/sulfamethoxazole, rifampicin, gentamycin, trimethoprim, metronidazole, ceftolozane, tazobactam, imipenem, cilastatin, relebactam and the like. In some embodiments, the anti-asthmatic agents, include, long and short acting B-agonists, including, formoterol, fluticasone, budesonide, mometasone, beclomethasone, ciclesonide, albuterol, ipratropium, theophylline, levalbuterol, and corticosteroids such as prednisone and methylprednisolone. In this and other embodiments, the diketopiperazine has the formula:

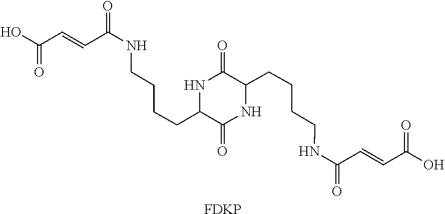 [SMILES: O=C(O)/C=C/C(=O)NCCCCC1NC(=O)C(CCCCNC(=O)/C=C/C(=O)O)NC1=O ; "FDKP"]

or a salt thereof, in particular, a disodium salt or a magnesium salt; wherein the diketopiperazine is in the form of a dry pharmaceutical crystalline powder, amorphous powder or forms microcrystalline particles suitable for inhalation. The pharmaceutical dry powders are administered to a patient in need with a dry powder inhaler in amounts up to 50 mg per dose in a single inhalation. In one embodiment, one or more doses of the dry powder pharmaceutical can be administered either independently for each active agent or combinations thereof. The powders can also be produced with one active agent or more than one active agent. In embodiments where the dry powder comprises a single active agent, the patient can be treated with one dose of each powder sequentially when needed. In some embodiments, the dry powders are manufactured comprising more than one active agent for the treatment of disease and the patient can be administered one or more than one dose of the dry powder combination.

The following examples illustrate some of the processes for making dry powders suitable for using with the inhalers described herein and data obtained from experiments using the dry powders.

### Example 1

Preparation of surfactant free dry powder comprising FDKP microcrystalline powder for use with inhalers: In an example embodiment, surfactant free dry-powders comprising FDKP microcrystalline particles were prepared. Using a dual-feed high shear mixer, approximately equal masses of FDKP solution (Table 1) and acetic acid solution (Table 2) held at about 25° C.±5° C. were fed at 2000 psi through a 0.001-in2 orifice to form a precipitate by homogenization. The precipitate was collected in deionized (DI) water of about equal temperature. The wt % content of FDKP microcrystallites in the suspension is about 2-3.5%. The suspension FDKP concentration can be assayed for solids content by an oven drying method. The FDKP microcrystallite suspension can be optionally washed by tangential flow filtration using deionized water. The FDKP microcrystallites can be optionally isolated by filtration, centrifugation, spray drying or lyophilization.

TABLE 1
Composition of FDKP Solution

| Component | Component Range (wt. %) |
|---|---|
| FDKP | 2.5-6.25 |
| 30% NH4OH Solution | 1.6-1.75 |
| Deionized Water | 92-95.9 |

TABLE 2
Composition of Acetic Acid Solution

| Component | Component Range (wt. %) |
|---|---|
| Acetic Acid | 10.5-13.0 |
| Deionized Water | 87.0-89.5 |

Dry powders (A, B, C and D) comprising microcrystalline particles made by the methods described above were tested for various characteristics, including surface area, water content and porosity measurements. Four different powders were used in this experiments. All powders tested had a residual water content of 0.4%. Table 3 demonstrates data obtained from the experiments.

TABLE 3

| | | Pore Volume | Pore Size |
|---|---|---|---|
| | Surface Area | BJH Adsorption | BJH Adsorption |
| | BET Surface | cumulative volume of | average pore |
| Powder ID | Area (m²/g) | pores (cm³/g) | diameter (4V/A) (nm) |
| A | 61.3 | 0.43 | 25.1 |
| B | 62.3 | 0.43 | 24.4 |
| C | 63.0 | 0.42 | 23.8 |
| D | 59.0 | 0.44 | 26.2 |

The data in Table 3 show that the surface area of sprayed-dried, bulk dry powder comprising the microcrystalline particles of the samples tested ranged from 59 m²/g to 63 m²/g. The porosity data indicate that the microcrystalline particles are relatively porous, having average pore volumes of about 0.43 cm³/g and average pore size ranging from about 23.8 nm to 26.2 nm as determined by BJH adsorption. In certain sample embodiments, the pore size of particles were up to 30 nm. The porosimetry data indicate that these particles differ from prior art FDKP microparticles, which have been shown to have an average pore volume of about 0.36 cm³/g and average pore size from about 20 nm to about 22.6 nm.

### Example 2

Preparation of dry powder comprising microcrystalline FDKP particles containing Δ9-THC or CBD. Isolated FDKP microcrystalline particles prepared as in Example 1 were suspended in ethyl alcohol. An approximately 1-4 wt % solution of Cannabis extract, primarily comprising either Δ9-THC or CBD, in ethanol and the ethanolic suspension of FDKP microcrystallites was added. Optionally, solutions of additives dissolved in ethanol were also added. The mixture was spray dried using a Buchi B290 spray-dryer equipped with a high efficiency cyclone. Nitrogen was used as the process gas (60 mm). Mixture were dried using 12-15% pump capacity, 70-100% aspiration rate, and an inlet temperature of 110-140° C. The weight % concentrations of Δ9-THC and additional additives are provided in Table 4. Delivery efficiencies of these powders after discharge from a dry powder inhaler ranged between approximately 50% and 70%.

TABLE 4
Composition of microcrystalline FDKP
particles containing Δ9-THC or CBD

| Component | Component Range (wt. %) |
|---|---|
| Δ9-THC and/or | 10-40 |
| CBD | |
| DPPC | 5-15 |
| DSPC | 5-15 |
| PVP | 0.5-5 |
| PEG | 2 |
| PS-80 | 2 |

Dry powders made by the method described above were tested using a substantially anatomically correct airway (ACA) system as described in U.S. Pat. No. 9,016,147. The dry powders exhibited significant degree of stability at room temperature, for example, at one-month storage, greater than 90% of the THC or CBD remained active with delivery efficiencies ranging from about 35% to about 75% using this method.

Table 5 illustrates data from triplicate sample of formulations made as described above containing up to 30% of the cannabinoid, THC with crystalline composite FDKP particles and various excipients components as indicated were tested in an ACA system. Powder samples were either hand-filled or filled into cartridges using a BioDot system. Cartridges were loaded onto a dry powder inhaler (MannKind Corp.), samples tested and the resultant data are shown in Table 5.

TABLE 5
ACA Performance with Processing Excipients, Hand
Filled vs. BioDot (1:1) Filled Samples

| | | | Hand filled | | BioDot Filled | |
|---|---|---|---|---|---|---|
| THC | | | | % | | % |
| content % | Excipient % | | % CE | Delivery | % CE | Delivery |
| 30% | 2% | PS 80 | 27.7% | 11.7% | | |
| 20% | 0.5% | PVP | 99.5% | 62.2% | | |
| 20% | 5% | PVP | 99.2% | 72.0% | | |
| 20% | 5% | PVP | 99.3% | 57.9% | 99.0% | 62.6% |
| 30% | 5% | PVP | 98.4% | 58.5% | | |
| 20% | 5% | PEG | 99.0% | 64.2% | 99.5% | 57.2% |
| 20% | 5% | DPPC | 99.0% | 59.9% | 98.7% | 60.3% |
| 30% | 10% | DPPC | 98.1% | 54.9% | 93.4% | 50.2% |

CE denotes cartridge emptying or % of total powder content emitted during testing of a batch.
Blanks indicate that the powders were not tested.

The data indicate that the powders made using various excipients have excellent aerodynamic characteristics as the resultant CE values are up to 99.5% of the total powder content in the inhalers were emitted. Thus the powders can be delivered effectively to the lungs as shown by the % delivery results of up to 72% of the powder content with the exception of the powder containing the surfactant PS80. It was observed that the formulation containing the PS 80 made the powder clumpy in this preparation. Powders made herewith appeared to be very stable as batches of the powders were tested at the time the powders were made by measuring the cannabinoid content and at two weeks and four weeks (room temperature storage) after the powder were made. Table 6 illustrates data from samples of powders showing cannabinoid content up to 4 weeks for various cannabinoid formulations.

TABLE 6
Chemical Stability

| | |
|---|---|
| % cannabinoid | % cannabinoid content (% assay remaining) |

| Formulation | Baseline | Two weeks | Four weeks |
|---|---|---|---|
| 20% THC | 11.20 | 12.71 (113.5%) | 11.90 (106.3%) |
| 30% THC | 20.41 | 20.95 (102.7%) | 19.16 (93.9%) |
| 20% CBD | 13.13 | 13.23 (100.8%) | 12.61 (96.0%) |
| 30% CBD | 20.04 | 21.75 (108.5%) | 22.16 (110.6%) |

The data in Table 6 indicate that the powder formulations made are very stable at room temperature, at least for the time period tested which is after 4 weeks of storage and for some samples the stability can be longer.

The preceding disclosures are illustrative embodiments. It should be appreciated by those of skill in the art that the devices, techniques and methods disclosed herein elucidate representative embodiments that function well in the practice of the present disclosure. However, those of skill in the art should, in light of the present disclosure, appreciate that many changes can be made in the specific embodiments that are disclosed and still obtain a like or similar result without departing from the spirit and scope of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope otherwise claimed. No language in the specification should be construed as indicating any non-claimed element essential to the practice of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments are described herein, including the best mode known to the inventors for carrying out the invention. Of course, variations on those preferred embodiments will become apparent to those of ordinary skill in the art upon reading the foregoing description. The inventor expects those of ordinary skill in the art to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

Furthermore, numerous references have been made to patents and printed publications throughout this specification. Each of the above cited references and printed publications are herein individually incorporated by reference in their entirety.

Further, it is to be understood that the embodiments disclosed herein are illustrative of the principles of the present invention. Other modifications that may be employed are within the scope of the invention. Thus, by way of example, but not of limitation, alternative configurations may be utilized in accordance with the teachings herein. Accordingly, the present invention is not limited to that precisely as shown and described.

We claim:

1. A dry powder pharmaceutical composition comprising microcrystalline particles of 3,6-bis(N-fumaryl-4-aminobutyl)-2,5-diketopiperazine and a cannabinoid; wherein the cannabinoid is in an amount from 1% to 40% (w/w) of the total weight of the composition.

2. The dry powder pharmaceutical composition of claim 1, wherein the cannabinoid is one or more of tetrahydrocannabinol, cannabidiol, or cannabinol, or combinations thereof.

3. The dry powder pharmaceutical composition of claim 1, wherein the dry powder further comprises a phospholipid selected from 1,2-dipalmitoyl-sn-glycero-3-phosphocholine and 1,2-distearoyl-sn-glycero-3-phosphocholine.

4. The dry powder pharmaceutical composition of claim 1, wherein the microcrystalline particles have an average pore volume of about 0.36 cm³/g to about 0.43 cm³/g.

5. The dry powder pharmaceutical composition of claim 1, wherein the microcrystalline particles have an average pore size from about 23 nm to about 28 nm.

6. The dry powder pharmaceutical composition of claim 1, wherein microcrystalline particles have a surface area of ranges from about 59 m²/g to about 63 m²/g.

7. The dry powder pharmaceutical composition of claim 1, wherein the microcrystalline particles have average pore volumes of about 0.43 cm³/g and average pore size ranging from about 23.8 nm to 26.2 nm as determined by BJH adsorption.

8. A dry powder inhaler comprising a dry powder pharmaceutical composition comprising microcrystalline particles of 3,6-bis(N-fumaryl-4-aminobutyl)-2,5-diketopiperazine comprising tetrahydrocannabinol, cannabidiol, or cannabinol in an amount from 1% to about 40% (w/w) of the total content of the dry powder.

9. The dry powder inhaler of claim 8, wherein the dry powder further comprises a phospholipid selected from 1,2-dipalmitoyl-sn-glycero-3-phosphocholine and 1,2-distearoyl-sn-glycero-3-phosphocholine in an amount up to about 15% (w/w) in the composition.

10. A method of treating chronic pain comprising, administering to a patient in need of treatment a therapeutically effective amount of the dry powder pharmaceutical composition of claim 1 by oral inhalation using a dry powder inhaler.

11. A dry powder drug delivery system comprising a dry powder inhaler comprising a dry powder pharmaceutical composition comprising microcrystalline particles of 3,6-bis(N-fumaryl-4-aminobutyl)-2,5-diketopiperazine and a cannabinoid; wherein the cannabinoid is in an amount from 1% to 40% (w/w) of the total weight of the composition and the microcrystalline particles have average pore volumes of about 0.43 cm³/g and average pore size ranging from about 23.8 nm to 26.2 nm as determined by BJH adsorption.
